(19) United States
(12) Patent Application Publication
(10) Pub. No.: US 2026/0325330 A1
(43) Pub. Date: Oct. 22, 2026

(54) Conformable Prostate Stent and Methods

(71) Applicants: William Joseph Drasler, Minnetonka, MN (US); William Joseph Drasler, II, Minnetonka, MN (US)

(72) Inventors: William Joseph Drasler, Minnetonka, MN (US); William Joseph Drasler, II, Minnetonka, MN (US)

(21) Appl. No.: 19/183,010

(22) Filed: Apr. 18, 2025

(51) Int. Cl. A61F 2/958

(52) U.S. Cl. CPC A61F 2/958; A61F 2210/0023; A61F 2210/0028; A61F 2210/0076

(57) ABSTRACT

A method for placing a stent into hollow member such as a prostatic urethra whereby the stent conforms to crevices located between prostatic lobes; the stent is able to heal and avoid encrustation and infection. The Nitinol stent is partially expanded in an elastic state into the prostatic lumen; the stent is then converted to a plastic state and further expanded into the crevices. The stent extends into the crevice forming an interlobular span that is covered by epithelium. The stent is converted back to an elastic state for implantation.

### BACKGROUND

Stents used to treat Benign Prostatic Hyperplasia (BPH) have encountered difficulties due in part to a lack of proper healing. Due to the generally tri-lobular shape of the prostatic urethra, crevices are created between the prostatic lobes, and the struts of a regular circular prostate stent are not able to make contact with the prostatic lobular tissues. The epithelial lining of the prostatic urethra is unable to extend onto the stent struts that subtend the span of the crevice resulting in a stent strut region that is prone to encrustation due to urinary salts and results in possible infection and luminal restriction.

Several prior art stent designs and materials have been tried for use as a prostate stent both for permanent implant and for temporary placement following an initial procedure to enlarge the prostatic lumen. One prior art prostate stent has been formed with two or more vertices that are formed into the stent's natural equilibrium shape; such stents require orientation to place the vertices in alignment with the prostatic crevice. However, prostatic lumens often have a variety of configurations and the locations of the prostatic crevices along the perimeter of the lumen are unique to each individual. Thus, a stent with a fixed shape, such as a triangular shape with three vertices, may not effectively place one of the vertices at the position of the prostatic crevice; this could result in a lack of proper healing and result in encrustation of the stent.

One material that is often used for both vascular and prostatic stents is Nitinol (NiTi). NiTi can be used as a superelastic material or alternately used as a temperature dependent or phase transition material. As a temperature dependent material, the use has involved delivery of the stent in a cooled state where the NiTi has a plastic state and can be formed into a small state. Exposure of the NiTi stent to a warmer body temperature after delivery to the prostatic urethra causes the NiTi to revert back to an enlarged elastic state that then holds the vessel lumen outwards at the diameter of the enlarged elastic state that is slightly larger than the lumen diameter of the vessel. This approach, however, does not require the stent struts to enter the prostatic crevice and hence is still subject to potential encrustation, infection, are prostatic lumen restriction.

Some prostatic stent are used mainly for temporarily support of the prostatic lumen and prevent luminal narrowing due to edema following a separate and different initial procedure to enlarge the prostatic lumen. Such stents are then removed after a few days or weeks via a secondary follow-up procedure.

What is needed is a permanently implanted prostatic stent that is able to extend into the prostatic crevices such that epithelium can extend from the prostatic lobes across the stent struts to avoid the effects of encrustation and infection. The prostatic stent should not require orientation in a circumferential direction and should extend into the prostatic crevices no matter where they are located. The prostatic stent in one embodiment should also have application as a temporary stent such that it can be removed easily via urethral access and not require a surgical procedure that causes the prostatic urethra to be surgically sectioned in order to remove the stent.

### SUMMARY

The present invention is a stent and methods of use to support a vessel, chamber, or hollow member of the body and has the capability of extending into cracks or crevices of the vessel, chamber, or hollow member that extend radially outwards from the lumen of the vessel, chamber, or hollow member to allow epithelial or endothelial cell coverage of the stent struts. The vessel, chamber, or hollow member can include the prostatic urethra located adjacent to the prostate tissues of the body. The stent is intended to enlarge the luminal diameter of the vessel or hollow member and provide improved healing of the stented luminal surface by making contact of the stent with surfaces within crevices that extend off of the vessel or hollow member lumen. The present invention includes a stent that has a plastic character so that the stent can be pushed into the crevices via a balloon, but after it has been expanded into the crevices, the stent has an elastic character and a large equilibrium diameter or controlled expansion diameter twice the vessel lumen diameter such that the stent remains in the crevices. The small distance that the stent struts span from one side of the crevice to the other allows the stent of this invention to obtain epithelial or endothelial coverage from the sides of the crevices. Such cellular coverage can provide enhanced healing of the stent leading to improved long term outcomes. The discussion found in this specification is focused on the use of the present invention in a prostatic urethra, however it is understood that the stent of the present invention can be applied to other vessels, chambers, or hollow members of the body including applications within blood vessels, cavities or chambers of the heart, and other chambers, cavities, and hollow members found in organs of the body.

The present invention has beneficial application as a prostate stent and methods of use; the prostate stent can be formed from NiTi with a temperature dependent character. When formed from NiTi, the stent is first partially expanded in an elastic state into contact with the lumen of the prostatic urethra. The stent is then converted to a plastic state and is expanded in a plastically deformable Martensitic state to expand the diameter of the lumen of the prostatic urethra and to extend the stent into the prostatic crevices formed by prostatic tissue lobes by an dilation balloon, and finally the stent is transformed or converted at body temperature back into an elastic Austenitic state as it is implanted into the prostatic crevices and into contact with the luminal surface of the prostatic urethra. As the stent is expanded into the prostatic crevices, the spanning distance that the stent struts extend from the surface of one prostatic lobe to the surface of a neighboring prostatic lobe within the crevice formed by the lobes is less than 2 mm such that epithelium growth can occur across the stent struts from one lobe to the next and obviate the potential issues associated with encrustation and infection. The prostatic stent can be initially positioned onto a dilation balloon and held onto the dilation balloon in an elastic, Austenitic state by a sheath. Upon the removal or retraction of the sheath, the stent can be allowed to expand outwards into contact with the prostatic lobes of the prostatic urethra in the Austenitic state. The stent can then be cooled by the dilation balloon to convert the stent into a plastically deformable Martensitic state for final expansion into the prostatic crevices.

Other methods for expanding the prostatic stent are also contemplated. For example, the prostatic stent can be converted to a plastically deformable Martensitic state while contained within the sheath. The cooled fluid from a dilation balloon can convert the stent from Austenitic to a plastically deformable Martensitic while the stent is positioned on the dilation balloon and held in a small diameter configuration within the sheath. The sheath can then be removed and the stent can be plastically expanded by the dilation balloon from its initial non-expanded Martensitic state on the dilation balloon into contact with the lumen of the prostatic urethra and crevices while remaining in a plastically deformable Martensitic state. After the stent has been expanded into the crevices, the stent can convert via exposure to body temperatures to an elastic Austenitic state as it becomes implanted.

The stent can have a zig zag stent structure that follows a helical path as it is positioned as a self-expanding stent in an Austenitic state on the outer surface of a dilation balloon and also is retained onto the dilation balloon by an outer sheath. The helical zig zag structure allows the stent to be easily removed from the prostatic urethra as potentially desired by pulling on a removal feature located on the proximal end of the stent member, and this feature allows the stent to be used either as a permanent stent or a temporary stent. Alternate structures for the stent are also contemplated. For example, the present stent can also be formed with a braided structure having several strands braided together to form a cylindrical structure. The braided stent can also be removed from the prostatic urethra as potentially needed by pulling on the proximal end while supporting the distal end with a distally directed force. Any stent structure used in the vascular or prostatic stenting industry can be used for the present invention, however not all structures lend themselves for ease of removal for temporary stent applications or removal for any other reason.

The stent of the present invention is formed with an equilibrium diameter (i.e., at body temperature and without constraint) in its Austenitic state that is at least twice the diameter of the prostatic lumen as defined by the closest distance between lobes that form the prostatic lumen. This significantly larger (i.e., larger than other standard prostatic stents) equilibrium diameter allows the stent of the present invention to extend into the prostatic crevices (after conversion to its plastically deformable Martensitic state) to a greater depth (or stent extension distance) such that the struts of the present stent span a distance from one lobe to a neighboring lobe that can be easily overgrown by epithelial cells. The stent's larger equilibrium diameter causes the stent to remain within the crevices in its elastic Austenitic state and not migrate out of the crevices toward the center of the prostatic urethral lumen. The span distance for the struts to allow for epithelial coverage is 1.5 mm (range 1-2 mm).

The stent of the present invention has a self-expanding or elastic structure in its final implanted Austenitic state so that the stent can alter its diameter in a region that is close to a sphincter muscle that changes in diameter significantly and often from day to day. Such sphincter muscles include the internal sphincter located between the bladder and the prostate and the external sphincter located between the prostate and the bulbous urethra.

The stent of the present invention is mounted onto an interventional catheter such as a dilation balloon located at the end of a balloon dilation catheter, for example. A sheath is placed over the stent in its elastic Austenitic state to hold the stent down into contact with the dilation balloon and is removed to release the stent into a partially expanded state that contacts the prostatic lumen in an elastic Austenitic state. The stent is partially expanded via self-expanding character into contact with the lumen of the prostatic urethra. The catheter shaft has a coolant lumen that extends from the catheter manifold to the dilation balloon. The coolant lumen is then used (after the stent has been released) to provide a cooled liquid such as saline or contrast medium, for example, from the catheter manifold to the inside of the dilation balloon after the dilation balloon is placed into contact with the luminal surface of the partially expanded stent. Inflation of the balloon with cooled fluid at a low balloon fill pressure places the stent into an Martensitic state that is a plastically deformable state. Further expansion of the dilation balloon at higher pressure expands the stent and enlarges the lumen of the prostatic urethra and expands the stent into the crevices.

As the catheter and stent are used, the dilation balloon and stent are first delivered to the site of the prostatic urethra, the sheath is pulled back and the stent is allowed to semi-expand or become partially expanded in its elastic (Austenitic) state as the stent comes into contact with the lobes of the prostatic lumen. The dilation balloon is then expanded into contact with the partially-expanded stent and the coolant liquid is delivered to the dilation balloon to place the balloon into contact with the stent and place the stent into a Martensitic state. The balloon is then further expanded to extend the stent while in a Martensitic state into the prostatic crevices to a stent extension distance extending outwards to a distance that is larger than the crevice opening distance extending off of the prostatic lumen; the crevice opening distance extending in a circumferential direction along the prostatic lumen from one lobe to a neighboring lobe. The opening distance is less than the diameter of the prostatic lumen and is often less than 50 percent of the prostatic lumen diameter. The coolant liquid is then terminated and the stent is allowed to heat to body temperature where it achieves the elastic state (Austenitic state) with an equilibrium diameter that can be at least twice the diameter of the prostatic lumen (the minimum lobe to lobe distance). The stent maintains its position within the crevices due to the expansion elasticity of the stent expanding outwards against the crevice and the surface of the prostate lumen; the stent struts exert outward forces to attain a larger stent equilibrium diameter which is larger than the prostatic lumen diameter including the surfaces of the crevices covered by the stent.

The inflation balloon can be removed by deflating the dilation balloon and applying tension to the catheter shaft. The catheter shaft can be braided to assist with providing improved torque transmission characteristics from the manifold to the distal end of the dilation balloon. Also, the catheter shaft can be rotated or torqued to help wind the balloon in a manner similar to its initial balloon winding direction. The prostatic stent of the present invention extending into the prostatic crevice has a span distance from one prostatic lobe to a neighboring lobe at the vertex or farthest edge of the stent entering into the crevice of approximately 1.5 mm allowing the dilation balloon to be released from the stent and removed from the urethra.

The stent can have a covering placed onto the struts. Such a covering can help ensure that the outward forces generated by the stent to hold the prostate lumen outwards do not allow the stent struts to cut and embed themselves too deeply into the prostatic tissues. Such a covering can be formed from an elastic polymer such as polyurethane or silicone, for example. Gaps or openings between helical winds of the covering can allow for epithelial growth onto the luminal surface of the stent covering and provide healing to the stent and covering to resist encrustation and potential infection. A drug, such as an anti-proliferative drug, for example, can be loaded into the coating and delivered to the prostatic tissues to help prevent excessive tissue ingrowth and potential prostatic lumen restriction.

### BRIEF DESCRIPTION OF THE DRAWINGS

FIG. 1A is a cross-sectional view of the prostatic lobes with a standard circular stent positioned within the triangular prostatic lumen.

FIG. 1B is a cross-sectional view of the prostatic lobes with a pre-formed triangular stent positioned within the triangular prostatic lumen.

FIG. 2A is a cross-sectional view of the prostatic lobes with the fully-expanded stent of the present invention extending into the interlobular crevices.

FIG. 2B is a side view of the prostatic stent positioned within the prostate tissue and within the prostatic urethra.

FIG. 3A is a plan view of the prostatic stent in an elastic state positioned on a dilation balloon and held in place by a sheath.

FIG. 3B is a prostatic stent formed from zig zag strut structure wound with a zig zag ribbon of struts a spiral manner that allow the stent to be removed from the body.

FIG. 3C is a plan view of a braided prostatic stent along with tools that allow the stent to be removed from the body.

FIG. 3D is a plan view of the prostatic stent having an upstream portion and a downstream portion that is more flexible than the central portion.

FIG. 3E is a cross-sectional view of a dilation balloon folded to form wings as the ballon is positioned on the tubular component of a balloon catheter.

FIG. 4A is a cross-sectional view of the prostatic lumen with a partially-expanded stent positioned therein and having a dilation balloon located within the stent lumen.

FIG. 4B is a side view of prostatic lumen with a partially-expanded stent positioned therein and having a dilation balloon located within the stent lumen.

FIG. 5A is a cross-sectional view of the partially expanded stent within the prostatic urethra being contacted by a cooled dilation balloon to convert the stent from an elastic to a plastic state.

FIG. 5B is a side view of the partially expanded stent contacted by a cooled dilation balloon to convert the stent from an elastic to a plastic state.

FIG. 6A is a cross-sectional view of the prostatic urethra with a dilation balloon expanding the stent into the crevices formed by the lobes of the prostate.

FIG. 6B is a side view of the dilation balloon expanding the stent outwards in a stent plastic state.

FIG. 7 is a side view of the stent positioned on a dilation balloon and held in place by a sheath while the stent is being cooled to form a plastic state.

FIG. 8 is a side view of the stent in an elastic state positioned on a central cylindrical member of a delivery catheter and held in place by a sheath for delivery to the prostatic urethra and release into the prostatic urethra in an elastic state.

### DETAILED DESCRIPTION

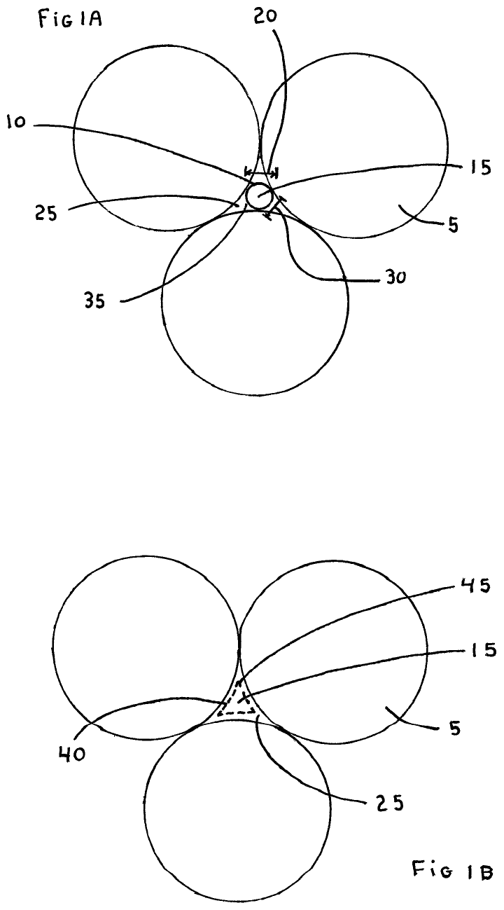

FIG. 1A shows three prostatic lobes 5 with a standard round cross-section stent 10 located in the prostate lumen 15 having a prostatic lumen diameter 20. The standard stent 10 (not the present invention) makes contact with a portion of each lobe but does not extend into the crevices 25 located between each of the lobes 5. The standard stent struts are required to cross a large span distance 30 (i.e., distance that the stent extends within the crevice from one lobe to another) greater than 2 mm that extends across the crevice opening 35. The epithelial cells from the adjacent lobes 5 cannot extend across the large span distance 30 and hence the stent struts are prone to encrustation, infection, and possible restriction of the prostatic lumen 15.

FIG. 1B shows a different prior art triangular stent 40 (not the present invention) having a pre-formed shape positioned within the prostatic lumen 15 and the crevices 25 that extend between neighboring lobes 5. This triangular stent 40 has its stent vertices 45 (the edge of the stent that extends farthest into the crevice) oriented such that they extend into each of the three crevices as shown here in an ideal prostatic cross-section. However, often the prostatic cross-section is not formed so ideally, but can instead take on a shape formed from primarily two lobes 5 or having one lobe that is significantly smaller than the other two lobes 5. In these instances, the triangular pre-shaped stent 40 can actually fit worse than a standard round cross-sectionally shaped stent 10. A vertex 45 of a pre-shaped 40 could easily be located in the center of a lobe rather than at a crevice, for example. Even in an ideal anatomy of a prostatic cross-section, the requirement of orienting the pre-shaped stent 40 to locate three vertices 45 adjacent to three crevices 25 is not desirable and not easy to visualize and achieve, thereby taking more time for the procedure.

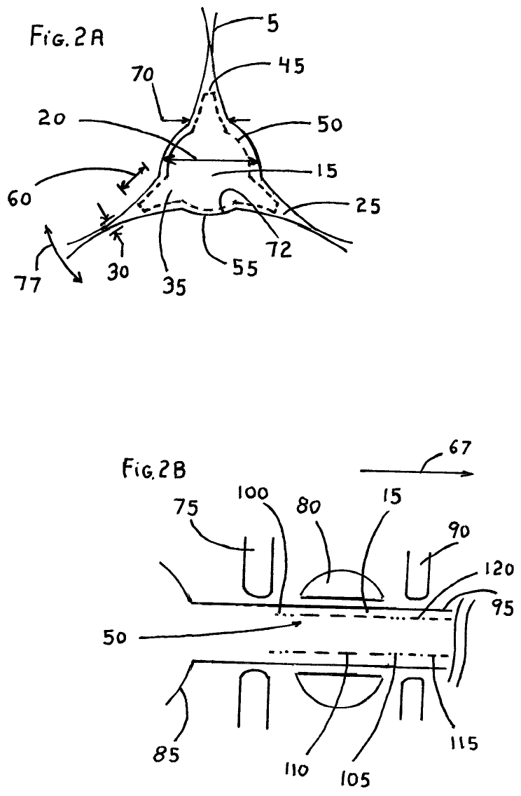

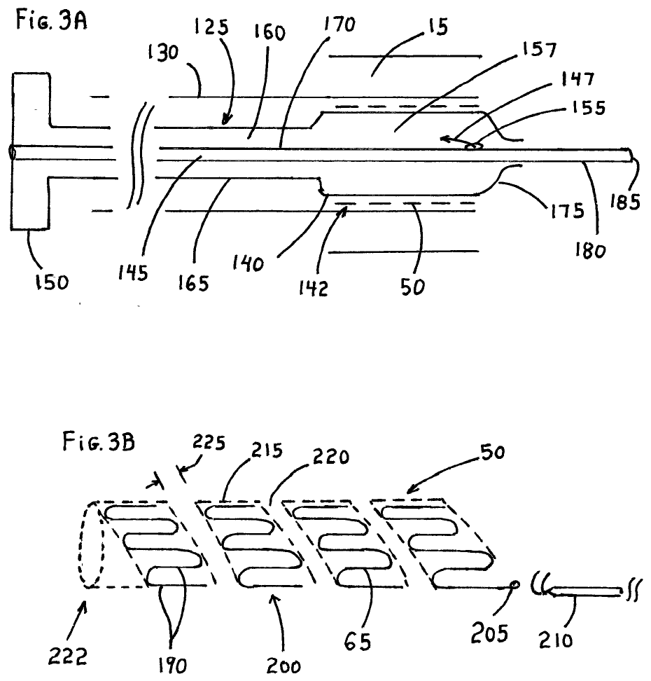

FIG. 2A shows a cross-sectional view of the prostatic stent 50 of the present invention in a final deployed configuration within the prostate lumen 15 and prostate crevices 25 between lobes 5 of the prostate 80. The lobes 5 create a crevice 25 that has a crevice opening 35 to the crevice of about one half the lumen diameter 20 of the prostatic lumen 15 as it extends from the lobe central luminal surface 55 to another lobe central luminal surface 55. The stent 50 has been expanded via a balloon 140 (see FIG. 3A) into the crevices 25 with the stent 50 in a plastically deformable Martensitic state. The stent 50 can extend in a radial direction into the crevice a stent extension distance 60 greater than the crevice opening distance 70. These stent extension portions 62 assist in preventing migration of the prostatic stent 50 in a downstream direction 67 (see FIG. 2A). The span distance 30 for the stent struts 65 within the crevice at the stent vertex 45 can be approximately one half of the crevice opening distance 70. The crevice opening distance 70 is less than the prostatic luminal diameter 20. If the prostatic lobes are circular and of equal diameter, the crevice opening distance 70 between lobes is 86 percent of the prostatic luminal diameter 20. Often the crevice opening distance 70 found in enlarged prostatic tissues is less than 50 percent of the prostatic lumen diameter 20. For a typical prostatic crevice extending off of a prostatic lumen 15 with a prostate lumen diameter of 6 mm, the crevice opening distance 70 can be 3 mm, the stent extension distance 60 can be 3 mm, the span distance 30 at the stent vertex, for example, can be 1.5 mm (range 1.0-2.0 millimeters) thereby allowing the epithelium from each lobe to migrate onto the stent struts 65 and prevent encrustation, infection, and prostatic lumen narrowing to occur. The stent 50 also is pressing outwards onto the center of the lobe central luminal surface 55 to provide an increased expanded prostatic luminal diameter 20 that is larger by 20-100% than the normal prostatic lumen diameter 20. The stent 50 of all embodiments of the present invention are intended to expand the diameter of the vessel, hollow member, or prostatic urethra luminal diameter 20 by 20-100%. The equilibrium diameter (in an unconstrained Austenitic state) for the present stent 50 invention is at least twice the prostatic luminal diameter prior to any expansion of the prostatic diameter.

The stent 50 of the present invention can also be applied to vessels or hollow members of the body that have crevices 25 that extend radially outwards from the main vessel lumen. The discussion in this specification will focus on a prostatic stent, however it is understood that the stent can be used in any chamber, vessel or hollow member of the body that has crevices that extend outwards in a generally radial direction. The present prostatic stent 50, for example, is configured to enter crevices 25 that have crevice openings 35 that are less than one half of the diameter of the vessel (i.e., range: one half to one quarter the vessel diameter). The present prostatic stent 50 is configured to make contact with the walls of the crevice such that the span distance 30 is 50% of the crevice opening distance 70 and extend into the crevice by a stent extension distance 60 that is greater than the crevice opening distance 70, the crevice opening distance extending in a circumferential direction 77 along the prostatic urethra luminal surface 72. For a prostatic urethra 135 with a prostatic lumen diameter 20 of 6 millimeters, for example, (range 4-9 millimeters), the crevice opening distance 70 can be approximately 3 millimeters. The present stent 50 is configured to make contact with the walls of the prostatic lobes 5 to form a span distance 30 of 1.5 mm (range: one to two millimeters); the span distance 30 is less than one third of the prostatic lumen diameter. The stent 50 extension is configured to extend, for example, into the crevice by 3 mm (range: two to four millimeters).

FIG. 2B shows a longitudinal view of the present prostatic stent 50 invention positioned within the prostatic lumen 15. An internal sphincter muscle 75 is located upstream of the prostate 80 to prevent or control urine flow out of the bladder 85; an external sphincter muscle 90 is located downstream of the prostate 80 and upstream of the bulbous urethra 95. To allow for constricting movement of the external sphincter 90 and internal sphincter 75, the prostatic stent 50 of the present invention can have an upstream portion 100 of approximately 3 mm (range 2-8mm) and/or a downstream portion 105 (range 2-8 mm) that can be formed of an elastic material having a more flexible structure than the remaining contiguous stent structure located in the central portion 110 of the prostatic stent 50. Such flexible stent strut structure 190 can be formed from stent struts 65 having a smaller strut width in the circumferential direction, smaller radial thickness, altered strut length (longer strut length, for example), reduced stent hinge width (i.e., located between stent struts 65), reduced thickness or material thickness between individual struts 65, altered (i.e., more flexible) metallic properties, or reduced stent diameter 250 including potential variable stent diameter 250 that can be a smaller diameter nearer to a sphincter.

The stent 50 of the present invention can also be connected with or joined to a holding member 115 that is located on one side of a sphincter that is opposite to the side of a sphincter to the prostate and adjacent to the prostate stent. The holding member 115 is connected to the prostatic stent 50 via a connecting element 120 that extends across one of the sphincter muscles. The holding member 115 is formed from an elastic material in its implanted state such as an elastic Nitinol, (for example) and can have a stent strut structure 190 similar to any portion of the prostatic stent 50 including the more flexible portions. The connecting element 120 can be one or more metal or polymeric filaments, for example; alternately the connecting element 120 can be a stent strut structure 190 similar to a portion of the prostatic stent 50 or formed from a structure similar to the prostatic stent 50 but more flexible (and can have an different stent diameter 250, such as a smaller stent diameter 250) than any portion of the prostatic stent 50; the connecting member connects the prostatic stent 50 to the holding member 115. The purpose of the holding member 115 is to prevent potential downstream or upstream migration of the prostatic stent 50. The prostatic stent 50 of the present invention, however, has stent extension portions 62 (see FIG. 2A) that extend into the crevices 25 located between each of the prostatic lobes 5 that help to prevent stent 50 migration in any direction. Frictional forces of the prostatic stent 50 against the prostatic lobe central luminal surface 55 also prevent migration of the prostatic stent 50 downstream or upstream. The holding member 115 and connecting element 120 are not a required feature of the present prostatic stent 50 invention.

FIGS. 3A-3E show the dilation balloon catheter 125 and sheath 130 that is used to deliver the prostatic stent 50 of the present invention through the prostatic urethra 135 from outside the body to the site of the prostatic lumen 15. As shown in FIG. 3A, the prostatic stent 50 is mounted onto a dilation balloon 140 located at the end of a balloon dilation catheter 125. The prostatic stent 50 is formed of a material that can change from a plastically deformable state to an elastically deformable state by altering its temperature. Such a material includes Nitinol (NiTi) for example, and other metal alloys and also some polymeric materials. The discussion found in this specification will focus on NiTi material, but it is understood that other materials including phase transitioning polymers can be used for the prostatic stent 50 of the present invention. As a further example, specific electroactive metal material can change their chemical state and stress-strain characteristics by application of electrical voltage to such a metal used as a stent material.

The NiTi prostatic stent 50, for example, can be loaded onto a collapsed dilation balloon 140 in its plastically deformable, Martensitic state at a temperature cooler than room temperature or body temperature. A thin sheath is advanced over the stent 50 to hold the stent 50 in a non-expanded stent configuration 142 onto the balloon 140 during the storage of the stent 50 and dilation catheter 125 at room temperature, for example, where the stent 50 has converted or transformed to an Austenitic or elastic state with a an equilibrium stent diameter 250 (in an unconstrained configuration at cooler than room temperature, for example, that is larger than twice the prostatic lumen diameter 20). Alternately, the NiTi prostatic stent 50, for example, can be loaded onto the dilation balloon 140 in an elastic, Austenitic state at a higher temperature, such as body temperature for the human body, or higher, for example, or at room temperature. After the stent 50 has been loaded onto the dilation balloon 140, the stent 50 is held onto the dilation balloon 140 by a thin sheath as shown in FIG. 3A. The diameter profile of the sheath is 2.5 mm (range 2-3.5 mm). In its Austenitic state, the prostatic stent 50 has a desire to expand out to an equilibrium stent diameter that is at least twice the prostatic lumen diameter 20 as measured across the prostatic lumen from one lobe 5 to another lobe 5 at the lobe central luminal surface 95.

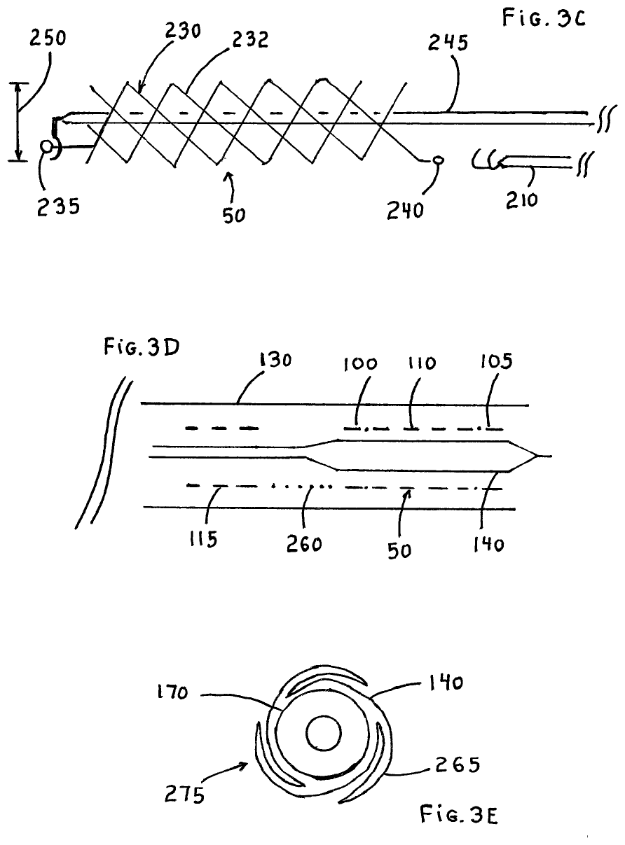

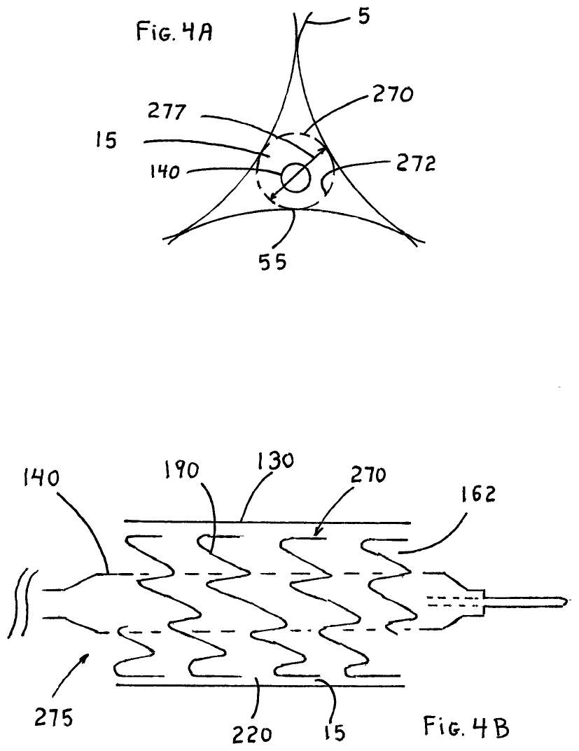

The dilation balloon catheter 125 has an inflation lumen 145 that provides inflation fluid 147 or inflation medium from the manifold 150 to a distal orifice 155 into the balloon lumen 157. The inflation fluid 147 can be saline or contrast medium, for example. The inflation fluid 147 can be cooled by a standard refrigeration system or via a heat exchanger positioned within an ice bath. A return lumen 160 located in the catheter shaft 165 is used to return cooled inflation fluid 147 from the inflation balloon 140 back to the manifold 150 to be re-cooled by the refrigeration system or unit and returned to the inflation balloon 140 as needed. The stent 50 can be cooled in one embodiment, for example, after the stent 50 has been released in an elastic, Austenitic state from the dilation catheter 125 by withdrawal of the sheath and the inflation balloon has been thereafter brought into the stent lumen 162 (see FIG. 4A) and inflated into contact with the luminal stent surface 272 (see FIG. 5A) to cool the stent 50 causing the stent 50 to convert to a plastically deformable, Martensitic state (see FIG. 4A).

The shaft 165 of the balloon catheter 125 can be braided to provide torque capability from the operator to the distal end 175 of the dilation balloon. A central tubular component 170 can reside within the balloon to deliver the inflation fluid; the tubular component 170 can extend from within the catheter shaft 165 or can be a portion of a multi-lumen catheter shaft 165 that extends within the dilation balloon from the catheter manifold 150 to the distal end 175 of the dilation balloon. This central tubular component 170 can provide the inflation fluid 147 to the balloon via the distal orifice 155. The central tubular component 170 can be a separate tubular member, for example, either metallic or polymeric, that extends through the catheter shaft 165 from the manifold 150 to a location within the distal end 175 of the dilation balloon and bonded to the distal end 175 of the dilation balloon. The tubular component 170 can extend distally past the balloon distal end 175 and serve as a fixed guide wire 180 that is fixed relative to the balloon. The hollow tubular member distal end 185 would be closed or blocked off such that the hollow tube could serve as both an inflation lumen 145 to inflate the inflation balloon and as a fixed guide wire 180. The tubular component 170 can be braided to provide improved torque transmitting character to the balloon distal end 175. It is understood that the balloon catheter 125 can alternately be an over-the-wire (OTW) catheter 125 similar to other over-the-wire balloon catheters 125. Such OTW catheters 125 can have one tubular component 170 extending through the catheter shaft 165 from the manifold 150 through the balloon to provide a guide wire lumen and have a second tubular component to provide an inflation lumen 145. A fixed wired catheter 125 has the capability of providing a lower diameter profile for improved entry and advancement through the prostatic urethra 135.

The stent 50 of the present invention can have a stent strut structure 190 similar to any vascular stent 50 or prostatic stent 50 found in the market or in the public domain. One advantageous stent 50 strut design is shown in FIG. 3B. The stent struts 65 can be folded back and forth into a zig zag stent strut structure 190, for example, that forms a stent ribbon 200; the stent ribbon 200 is then wound into a spiral shape to form a round cylindrical stent 222 having a circular cross-section. The stent strut thickness in a radial direction is 0.004 inches (range 0.025-0.007 inches). The stent diameter 250 of stent 50 in its non-expanded configuration 142 is 2 mm (range 1.5-3 mm) and the prostatic stent 50 is able to extend out to support a prostatic lumen 15 of 6 mm (range 3-8 mm) and extend out into crevices 25 by a stent extension distance 60 of 3 mm. This stent strut structure 190 offers an opportunity to be used as a temporary stent 50 or as a permanent stent 50. As a permanent stent 50, its intent is to be implanted into the prostate lumen 15 for a long period of time. However, if there is a need to remove the stent 50 due to poor positioning, migration, the presence of pain, or other reason, the stent 50 can be removed by grasping a removal feature 205 (such as a loop or geometric member that can be grasped by a tool, see FIG. 3B) located at the downstream end of the stent 50 with a grasping tool 210; the grasping tool can be contained within a removable protective sheath for introduction and delivery into the urethra 135. The grasping tool 210 is then pulled downstream under tension within the urethra 135 and the stent 50 is allowed to unwind the spiral ribbon 200 and unfold the zig zag stent strut structure 190 into a linear configuration of the stent struts 65 to be removed from the urethra 135. Both the zig zag stent strut structure 190 and the spiral stent ribbon 200 are configured to unfold and unwind, respectively, to form a linear configuration. The stent ribbon 200 can have a thin coating 215 (0.0005-0.002 inch thick) of a flexible polymer that weakly attaches the zig zag stent strut structure and can hold the stent struts together into a stent ribbon 200. The presence of such a coating 215 prevents the stent struts 65 from embedding themselves too deeply into the surface of the prostatic lobes 5 and keeps the struts 65 localized near the surface (i.e., 0005-0.050 inches from the lobe central luminal surface 55) of the prostatic lobes 5. The coating 215 helps to allow the struts 65 to transmit an outward force of the stent 50 to the prostatic lobes 5 to open the prostatic lumen 15 to a larger prostatic lumen diameter 20. The coating 215 can contain a drug that can help to reduce cellular hyperplasia due in part to excessive expansion of the prostatic lobes 5, for example. Other drugs can also be loaded into the coating 215 for diffusion into the surrounding tissues to fight infection, for example, or for other purposes. A gap 220 can be located between each helical ribbon 200 as it spirals around into a cylindrical stent 222 that forms the prostatic stent 50. The gap 220 can provide a space for cellular ingrowth from the lobular epithelium onto the luminal surface of the stent 50 providing a healing benefit that resists the formation of encrustation and possible infection or stent 50 restriction. The gap 220 can range in gap width 225 from 0.0005 inch to 0.040 inch or greater. As a temporary stent 50, the stent 50 can be removed after the patient has had another, separate, and previous, procedure to enlarge the prostatic lumen 15 and it is necessary to maintain the prostatic lumen 15 for several days or weeks until the edema from the other procedure has subsided. The temporary stent 50 can have a similar coating 215 and removal feature 205 as found on the permanent stent 50.

Another stent structure 230 of the present invention is shown in FIG. 3C. The prostatic stent 50 can have a braided stent structure 230 formed from 6 strands 232 (range 4-8 strands 232) of thermally modifiable or temperature dependent (i.e., strands can be altered from plastic to elastic state) material such as NiTi, for example.

The method of loading the stent 50 onto the dilation balloon is similar to that described above for other stent structure or designs. The braided stent structure 230 can also be used for either a permanent stent 50 or a temporary stent 50. A downstream removal feature 240 located at the downstream end of the stent 50 can be releasably attached to a grasping tool 210 that is entered into the urethral lumen at a time that it is desired to remove the stent 50. The downstream removal feature 240 can be pulled distally by the grasping tool 210 to place the stent 50 into tension and reduce the stent diameter 250 and remove the stent 50 downstream through the urethra 135. Also, an upstream removal feature 235 can be located at the upstream end of the stent 50. A compression tool 245 (such as a solid incompressible, flexible rod with a grasping fixture, for example) can be entered into the urethra 135 an releasably attached to the upstream removal feature 235 to apply under compression a force to the upstream end of the stent 50 to place the stent 50 into tension. Application of an upstream compression force from the compression tool 245 directed in an upstream direction along with a grasping tool 210 applying a downstream tensional force will cause the braided stent 50 to reduce in stent diameter 250 as the stent 50 becomes elongated in length and able to be removed downstream via the urethra 135.

As shown if FIG. 3D and previously described earlier in FIG. 2B, the prostatic stent 50 of the present invention can have an upstream portion 100 and a downstream portion 105 that is more flexible than the central portion 110 and is formed from a material having an elastic state in its final implanted configuration at body temperature. The purpose of this more flexible and elastic upstream or downstream portion 105 is to allow the stent 50 to change stent diameter 250 easily without affecting the ability of the internal sphincter 75 or external sphincter 90 to contract or expand naturally. For a zig zag stent strut structure 190 (see FIG. 3B), the stent struts 65 for the upstream portion 100 or downstream portion 105 can be reduced in diameter (via centerless grinding, for example) prior to forming a more flexible zig zag shape. For the braided stent structure 230 (see FIG. 3C) the braided stent structure 230 can be placed onto a mandrel and the braided strands 232 in the upstream portion 100 can be mechanically flattened to a thinner radial dimension, centerless ground to a thinner radial dimension, or thermally altered to alter their crystalline state to make the upstream portion 100 or downstream portion 105 weaker or more flexible in diametric flexation than the central portion 110. The prostate stent 50 of the present invention can have a holding member 115 located on the opposing side of the external sphincter 90, for example, from the prostatic stent 50 and the holding member 115 can be attached to or contiguous with the prostatic stent 50. The holding member 115 can be formed from the same material and have the same shape, diameter, and flexibility of any of the embodiments described for the stent in this specification or found in the literature or public domain. The holding member 115 can be attached to the prostatic stent 50 via a connecting element 120 such as a fiber or a wire, for example. Alternately, the holding member 115 can be contiguous with the stent 50 and have a stent structure 190 that is the same or similar to either the stent 50 upstream portion 100, downstream portion 105, or central portion 110. The holding member can help to hold the prostatic stent from migration downstream within the urethra 135.

During delivery and use of the stent, the circular cross-section prostatic stent 50 of the present invention is released from the dilation catheter 125 by retracting the sheath 130 and the stent 50 undergoes a partial-expansion to form a circular shape in contact with lobe central luminal surfaces 55 of the prostatic lobes 5 (Described further in FIG. 4A). The dilation balloon 140 was located radially adjacent to the prostatic stent 50 in its initial balloon non-expanded configuration 275; the balloon can be wound into a circle with wings 265 wound around the tubular component 170 similar to that shown in FIG. 3E. Following the release of the stent 50 from the wings 260 of the dilation balloon 140 via withdrawal of the sheath 130, the stent 50 undergoes an elastic partial expansion into contact with the lobe central luminal surface 55 of the prostatic urethra 135. The dilation balloon is inflated at low pressure (range 0.5-2 atm) to open up the wings 265 and place the dilation balloon 140 into contact with the lobe central luminal surfaces 55 of the prostatic lobes 5 (see FIG. 4A) such that the dilation balloon 140 has the same diameter as the prostatic luminal diameter 20. The inflation medium 147 is in a cooled state such that the cooled balloon contact with the stent luminal surface 272 of the partially expanded stent 270 converts or transforms the stent 50 into a plastically deformable, Martensitic, state. The balloon can be formed from a material that is semi-compliant such that it can reach a diameter of twice the prostatic lumen diameter 20 at a pressure of 7 atm (range 5 -15 atm) due to uniaxial or biaxial stretching of the balloon material. Alternately the balloon can be formed from a non-compliant material that has a diameter at 7 atm (range 5 -15 atm) that is twice the prostate lumen diameter, and the balloon acts as an enlarged bag to push into the crevices 25 due to its larger equilibrium diameter (i.e., balloon in a native state without an internal expanding force, but with a small filling pressure of approximately 0.1-2 atm.). A compliant balloon can also be used for the inflation balloon provided that the pressure vs diameter sizing are known such that the stent 50 is not pushed outwards into the crevices 25 until after the stent 50 has reached the Martensitic state (or cooling of the inflation medium 147 with the balloon). A compliant balloon can expand out uniformly via biaxial expansion and does is not required to be folded with wings 265 to obtain a small diameter non-expanded configuration 275.

FIGS. 4A and 4B show the partially expanded stent 270 after it has been released by retraction of the sheath 130 and has come into direct contact with the lobe central luminal surfaces 55 of each of the lobes 5 of the prostatic lumen 15. Releasing the stent 50 by withdrawing the sheath 130 allows the elastically deformable stent 50 in its elastic Austenitic state to expand uniformly into contact with the lobe central luminal surface 55. The zig zag stent strut structure 190 has opened up partially and the spirally wound ribbon 200 has unwound partially providing a larger gap than the initial gap 220. The dilation balloon 140 is positioned within the stent lumen 162 and has not yet been inflated and has a smaller balloon non-expanded configuration 275 with the balloon at a smaller diameter than that of the stent partially-expanded diameter 277. The prostatic stent 50 in its elastic, Austenitic state of the present embodiment is then expanded uniformly along its perimeter by the expansion of the dilation balloon. It is noted that a holding member 115 (see FIGS. 2B and 3D) that can be connected via the connecting element 120 to the prostatic stent 50 can also be positioned within the sheath 130 and can be released by withdrawal of the sheath 130 in a proximal direction. The holding member 115 and connecting element 120 are not positioned radially outward from the dilation balloon and therefore the holding member 115 can have an elastic state, and also remain elastic during implantation.

After the stent 50 has been released and partially expanded into contact with the prostatic lumen 15 and the dilation balloon 140 is positioned within the stent lumen 162, the dilation balloon 140 can be inflated to a balloon fill pressure of 0.3-3 atm. to form a balloon partially-expanded configuration 280 via inflation with a cooled fluid 185 such as cooled saline or cooled contrast medium, for example as shown in FIGS. 5A and 5B. The cooled fluid 185 can be provided by a refrigeration unit 287 that supplies the cooled fluid 185 via the catheter manifold 150 to the inflation lumen 145 and into the dilation balloon via a distal orifice 155 located in a central tubular component 170. A return lumen 160 returns the inflation fluid 147 through the catheter shaft 165 to the manifold 150 where the return fluid is directed back to the refrigeration unit 287 to be further cooled and returned to the inflation balloon at a temperature below that needed to cause the NiTi stent 50 to be transformed into a Martensitic state. As the dilation balloon expands outward to a balloon partially-expanded configuration 280 at low fill pressure (range 0.5-3 atm) into contact with the stent luminal surface 272 of the partially-expanded stent 270, the cooled inflation balloon causes the partially expanded stent 270 to convert or transform to a Martensitic state with plastically deformable properties. The inflation balloon may provide contact with the stent 50 for several minutes to ensure that conversion to the Martensitic state is complete.

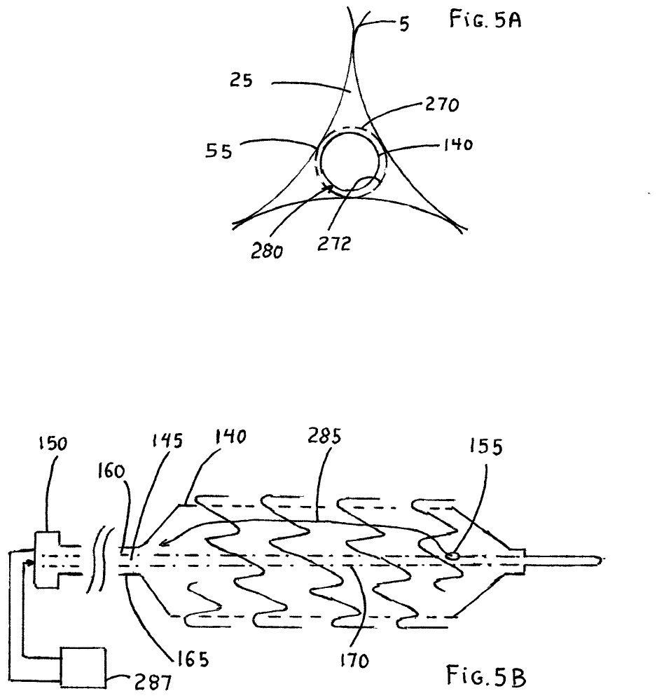

After the stent 50 has been converted to a plastically deformable Martensitic state, the dilation balloon 140 can be inflated further to a fully-inflated balloon configuration 295 at a higher pressure of 7 atm (range 5-15 atm) via the cooling fluid to cause the stent 50 to easily deform and enter into the crevices 25 as shown in FIGS. 6A and 6B. The dilation balloon is expanded to a fully-expanded balloon diameter 300 (i.e., a balloon diameter required to cause the stent to enter the crevice and achieve a proper stent extension distance 60). The stent 50, which can have an equilibrium diameter (non-constrained and in an elastic, Austenitic state) of at least twice the prostatic lumen diameter 20 can enter the crevices 25 for a stent extension distance 60 that is greater than the crevice opening distance 70 and reach a span of less than 1.5 mm (or less than 2 mm) at the stent vertex 45 of the stent extension portion 62. The stent extension portions 62 protrude into the crevice by a stent extension distance 60 and extend outward in a radial direction. As the stent 50 enters the crevices 25, the stent 50 strut span from one prostatic lobe to a neighboring lobe is reduced and the span becomes less than 2 mm at the stent vertex 45. The epithelium from each of the neighboring lobes 5 is able to migrate and grow on the stent struts 65 or the stent coating 215 for a small span distance 30 of 1.5 mm to provide a cellular coating 215 that resists the formation of encrustation and possible infection or narrowing of the prostatic lumen diameter 20 as described as extending from one central lobe surface to another. Following dilation of the dilation balloon to a fully-expanded balloon configuration 295 and full expansion of the stent 50 to a fully-expanded stent configuration 305 into the crevices 25, the cooled inflation fluid 147 need not be further cooled, and the stent 50 can be allowed to revert to an elastic Austenitic state at body temperature. Since the stent 50 has a stent equilibrium diameter (in free space without any constraint) in an elastic Austenitic state that is at least twice the prostatic lumen diameter 20, the stent extension portions 62 that extend into the crevices 25 will have a natural tendency to remain in the crevices 25 when the stent 50 is converted to the Austenitic state.

After the stent 50 has been expanded into the crevices 25, the inflation balloon can be deflated and removed from the prostatic lumen 15 and crevices 25 leaving the stent extension portions 62 located within the crevices 25 as shown in FIG. 6A. The deflation of the balloon can occur either with the stent 50 still in its plastically deformable Martensitic state or after the stent 50 has been warmed (with the balloon still expanded into the crevices 25) under body temperature or via introduction of warm inflation fluid 147 (i.e., inflation fluid 147 can be at room temperature, body temperature or slightly warmer than body temperature, for example) into the inflation balloon. With the stent 50 converted to an elastic Austenitic state, the stent extension portions 62 located at the stent vertex 45 has a tendency (due to the cylindrical equilibrium shape of the stent 50) to release from the inflation balloon and allow for ease of removal of the balloon from the stent 50 located deep within the crevices 25. The balloon catheter 125 can be torqued or turned in a direction that is favorable to refold the balloon wings 265, for example, to assist in removing the balloon from the crevices 25; vacuum can be applied to the inflation lumen 145 to reduce the profile of the inflation balloon to assist in removal of the balloon catheter 125 from the urethra 135. An external sheath 130 such as the initial sheath 130 that was used to hold the stent 50 onto the inflation balloon can be used to assist in removal of the deflated inflation balloon (i.e., pull the inflation balloon into the sheath 130) after delivery of the stent 50 to the prostatic lumen 15 and crevices 25. To place the sheath 130 back over the dilation balloon 140, the sheath 130 can be introduced over the catheter shaft 165 to a location just downstream of the proximal end of the dilation balloon (note that the balloon catheter shaft 165 can be long enough to allow the sheath 130 to reside over the catheter shaft 165 outside of the urethra 135 and distal to the catheter manifold 150). The dilation balloon can be then pulled into the distal end of the sheath 130 and both the sheath 130 and the balloon catheter 125 can be removed together out of the urethra 135 following delivery of the stent 50 to the prostatic urethra 135 and crevices 25.

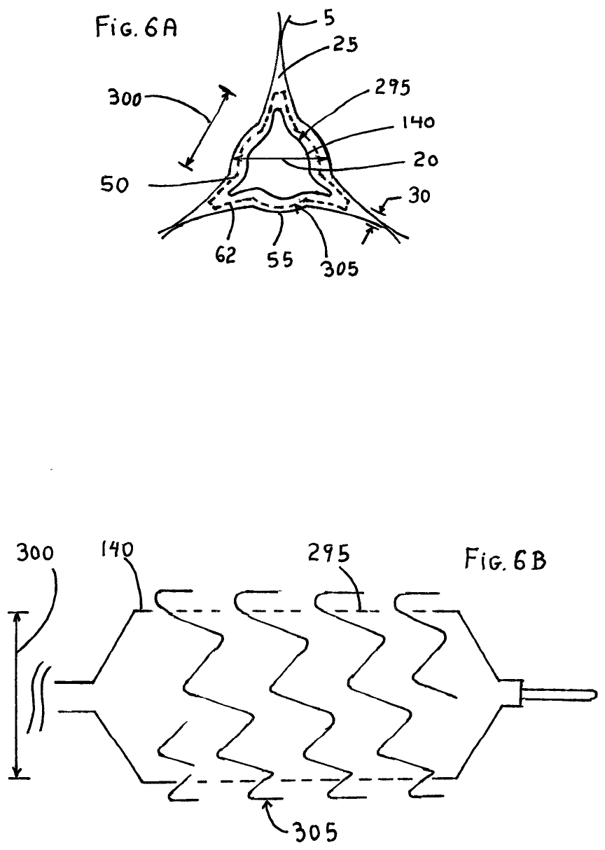

An alternate method of use for the present invention is shown in FIG. 7. The prostatic stent 50 has been delivered to the prostatic lumen 15 with the stent 50 held onto a dilation balloon by a sheath 130. The refrigeration unit 287 generates cooled fluid 185 that is delivered out of the distal orifice 155 with the sheath 130 still covering the prostatic stent 50 as shown in FIG. 7. The prostatic stent 50 is converted using cooled fluid 185 from the dilation balloon from an elastic Austenitic state (as the stent 50, dilation catheter 125, and sheath 130 were stored at room temperature or warmer temperature, for example) to a plastically deformable Martensitic state with the sheath 130 covering the stent 50 and the dilation balloon in a balloon non-expanded configuration 275. The ability to deliver cooling fluid to the balloon such that the cooling fluid can cool the stent 50 is not an efficient as previously described in FIG. 5B where the dilation balloon is in a balloon partially-expanded configuration 280. The resistance to the flow of cooling fluid in the embodiment of FIG. 7 is greater due to the presence of the unexpanded material of the dilation balloon and as a result the conversion of the stent 50 to a Martensitic state would take a longer time. A higher pressure (range 2-5 atm) can be applied by the refrigeration unit 287 to increase the flow rate of cooled fluid 185 out of the distal orifice 155 and reduce the time to achieve a conversion to the Martensitic state.

Once the prostatic stent 50 has been converted to the Martensitic state, the sheath 130 is then withdrawn and the dilation balloon can dilate the prostatic stent 50 in a plastically deformable state to expand the prostatic stent 50 in a continuous step (from the initial configuration of the stent contained within the sheath to its fully-expanded configuration 295) into the crevices 25 as previously shown in FIGS. 6A and 6B. The dilation balloon can be inflated at pressures ranging from 5 -15 atm to place the stent 50 extensions into the crevices 25 and to dilate the prostatic lobe central luminal surface 55 in an amount ranging from 20-100% greater than the initial prostatic lumen diameter 20. Following delivery of the prostatic stent 50 into the crevices 25, the coolant fluid used to inflate the balloon is no longer cooled and the prostatic stent 50 is allowed to warm to body temperature and convert to an elastic, Austenitic state. The dilation balloon can then be removed from the prostatic urethra 135 and removed from the patient's body.

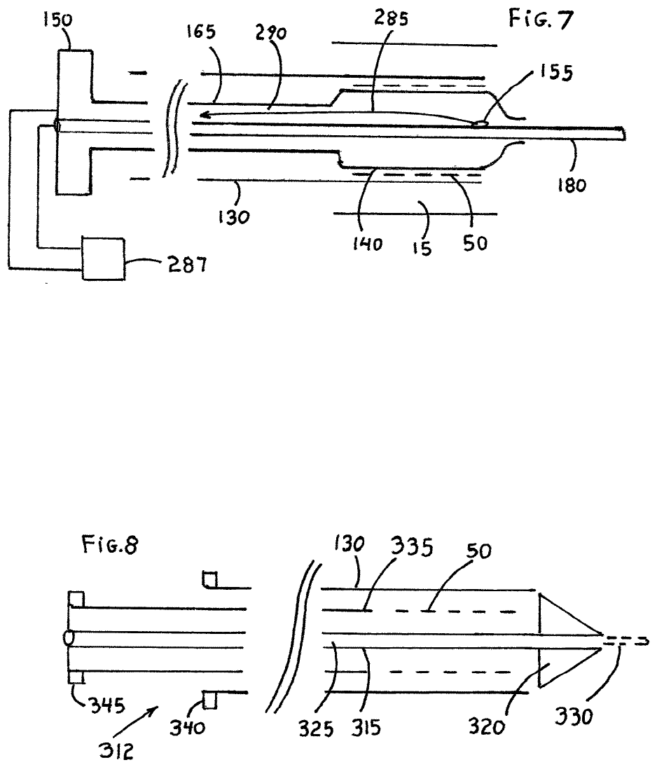

Yet another embodiment for the prostatic stent 50 of the present invention is shown in FIG. 8 with the stent 50 being delivered to the prostatic lumen 20 via an interventional catheter such as the delivery catheter 312, for example. The prostatic stent 50 is mounted on a central cylindrical member 315 that is permanently attached distally to a conical tip 320. The conical tip 320 makes a smooth closure to the distal end of a sheath 130; the sheath 130 holds the prostatic stent 50 into contact with the central cylindrical member 315. The conical tip 320 provides a smooth entry for the sheath 130 into the urethra 135 of the individual and for passage within the urethra 135 to the prostatic urethra adjacent to the prostate 80. The central cylindrical member 315 can be a hollow tube with a central lumen 325 for passage of a guidewire 330 therethrough; alternately the central cylindrical member 315 can be a solid cylindrical member. The solid cylindrical member can extend through the tip and beyond the conical tip 320 to serve as a fixed guidewire 330 to assist in passage of the sheath 130 through the urethra 135, or alternately a separate fixed guidewire can be attached to the conical tip 320.

After the sheath 130 has been placed within the prostatic urethra 135, the sheath 130 can be withdrawn backwards relative to a pusher 335 by pulling back on the sheath manifold 340 while retaining the pusher manifold 345 in place. The prostatic stent 50 is then free to expand outwards via its elastic Austenitic state as described earlier for the prostatic stent 50 that is at a warm temperature similar to room or body temperature. The prostatic stent 50 expands out elastically to a partially-expanded stent 270 into contact with the lobe central luminal surface 55 as described earlier. The sheath 130 and pusher 335 can then be removed from the prostatic urethra 135 and removed from the body.

In this embodiment as shown in FIG. 8, a separate balloon catheter 125 with a dilation balloon 140 is then entered into the urethra 135 and the dilation balloon is advanced to the prostatic urethra 135 and into the central lumen of the partially-expanded stent 270 as shown in FIGS. 5A and 5B. The dilation balloon is expanded in a manner similar to that described for FIGS. 5A and 5B using a cooling fluid as shown in FIGS. 5A and 5B. The dilation balloon is placed into contact with the prostatic stent 50 at a low pressure ranging from 0.5 to 3 atmospheres for a period of several minutes and the cooled fluid 185 in the dilation balloon causes the prostatic stent 50 to convert to a plastically deformable Martensitic state. The dilation balloon pressure is then increased to 7 atm (range 5-15 atm) to push the prostatic stent 50 into a fully expanded stent configuration 305 that is expanded into the crevices 25 and form a span of 1.5 mm as shown and described in FIGS. 6A and 6B. The balloon materials and stent 50 materials of this embodiment are the same as that described for earlier embodiments of the present invention. Cooling to the dilation balloon is terminated after the stent 50 extensions have been extended into the crevices 25 for a stent extension distance 60 greater than the crevice opening distance 70. The dilation balloon is then removed from the prostatic urethra 135 and from the patient's body.

Reference numerals found in drawings of an embodiment can be equally applied to other embodiments and bear the same meaning.

1. The method for expanding a hollow member of a body to a larger hollow member diameter via placement of a stent, said stent being able to convert from an elastic state to a plastic state and back to an elastic state, the hollow member having a crevice that extends radially outwards from a hollow member lumen, the crevice having a crevice opening along a hollow member luminal surface, the crevice opening having a crevice opening distance less than a hollow member lumen diameter, the method comprising, in order, the steps;
A. partially expanding said stent in said elastic state, said stent expanding into contact with the hollow member luminal surface,
B. converting said stent to said plastic state,
C. further expanding said stent in said plastic state, said further expanding comprising inflating a dilation balloon positioned on a distal end of a balloon dilation catheter to expand the hollow member lumen to the larger hollow member lumen diameter, and to extend said stent into the crevice to improve healing of said stent, said stent extending into said crevice by a stent extension distance,
D. converting said stent back to said elastic state and implanting said stent into the hollow member, said stent remaining extended into the crevice by said stent extension distance.

2. The method of claim 1 wherein said stent extends within said crevice by said stent extension distance that is greater than the crevice opening.

3. The method of claim 1 wherein said stent has an equilibrium diameter that is two times larger than the hollow member lumen diameter thereby causing said stent to remain extended within the crevice for said stent extension distance.

4. The method of claim 1 further comprising the step of removing said balloon dilation catheter from the hollow member of the body.

5. The method of claim 1 further comprising the step of removing said stent from the hollow member of the body by grasping a distal end of said stent and placing said stent under tension.

6. The method of claim 1 wherein said partially expanding said stent in said elastic state step comprises retracting a sheath that holds said stent in an unexpanded configuration onto an interventional catheter, said retracting a sheath allowing said stent to partially expand in an elastic state into contact with the hollow member luminal surface.

7. The method of claim 1 wherein said step of converting said stent to said plastic state comprises inflating said dilation balloon into contact with said partially expanded stent and cooling said stent via cooled fluid delivered to said dilation balloon from said balloon dilation catheter thereby causing said partially expanded stent to be converted from said elastic state to said plastic state.

8. The method of claim 1 wherein the step of further expanding the stent in said plastic state comprises further inflating said dilation balloon within a stent lumen with said stent in a plastic state to cause said stent and the hollow member lumen diameter to enlarge and cause said stent to extend into the crevice.

9. The method of claim 6 wherein said interventional catheter is said balloon dilation catheter.

10. The method of claim 1 wherein said hollow member is a prostatic urethra.

11. The method of claim 8 wherein said crevice is formed from contact of two lobes of a prostate tissue.

12. The method of claim 1 wherein said dilation balloon has a fully inflated diameter that is at least twice the hollow member lumen diameter.

13. The method for expanding a hollow member of a body to a larger hollow member diameter via placement of a stent, said stent being able to convert from an elastic state to a plastic state and back to an elastic state, the hollow member having a crevice that extends radially outwards from a hollow member lumen, the crevice having a crevice opening in a hollow member luminal surface, the crevice opening having a crevice opening distance less than a hollow member luminal diameter, the method comprising, in order, the steps;
A. delivering a stent in an elastic state mounted on the outside of a dilation balloon and positioning said stent adjacent to the hollow member lumen to be expanded,
B. retracting a sheath from a position on the outside of said stent to release said stent in an elastic state from an non-expanded configuration to a partially expanded configuration into contact with the hollow member lumen,
C. inflating said dilation balloon into contact with said stent with said stent in said partially expanded configuration, said dilation balloon being inflated with a cooled fluid,
D. converting said stent from an elastic state to a plastic state by cooling said stent due to said contact with said dilation balloon that is inflated with said cooled fluid,
E. further inflating said dilation balloon, said further inflating said balloon comprising inflating with said cooled fluid to expand said stent in a plastic state and expand the hollow member lumen to a larger diameter, said stent extending into the crevice by an stent extension distance that is greater than the crevice opening.

14. The method of claim 13, following said further inflating said dilation balloon step, comprising the step of warming said stent to convert said stent back from a plastic state to said elastic state, said stent remaining extended into the crevice.

15. The method of claim 14 further comprising the step of removing said dilation balloon from the hollow member of the body.

16. The method of claim 13 wherein said hollow member of the body is a prostatic urethra.

17. The method of claim 16 wherein said stent is removed from the prostatic urethra by application of tension to a proximal end of said stent.

18. The method for expanding a prostatic urethra to a larger radial dimension via placement of a stent, the prostatic urethra having crevices formed between prostate tissue lobes, the crevices extending radially outwards from a prostatic urethral lumen, the crevices having a crevice openings with crevice opening distances extending along a prostatic urethral luminal surface between the prostatic tissue lobes, the method comprising the steps;
A. releasing a stent from an interventional catheter and allowing said stent to partially expand in an elastic state into contact with the prostatic urethral luminal surface,
B. converting said stent to a plastic state by exposing said stent to a dilation balloon that is cooled by a
cooled fluid, said dilation balloon converting said stent from said elastic state to said plastic state,
C. further expanding said stent in said plastic state with said dilation balloon inflated by said cooled fluid to expand the lumen of the prostatic urethra, and to expand said stent in a plastic state into the crevice by a stent extension distance.

19. The method of claim 18 wherein said stent is converted back to said elastic state by no longer applying said cooled fluid to said dilation balloon thereby allowing said stent to warm to body temperature and convert back to said elastic state.

20. The method of claim 18 wherein said stent extension distance is greater than the crevice opening distance.
